(19) United States
(12) Patent Application Publication
(10) Pub. No.: US 2009/0018503 A1
(43) Pub. Date: Jan. 15, 2009

(54) SINGLE USE SAFETY SYRINGE HAVING A RETRACTABLE NEEDLE

(75) Inventors: Graeme Francis Walton, New South Wales (AU); Allan Walsh, New South Wales (AU); Zuo Qian Lin, Zhejiang Provence (CN)

Correspondence Address: BROOKS KUSHMAN P.C., 1000 TOWN CENTER, TWENTY-SECOND FLOOR, SOUTHFIELD, MI, 48075, US

(73) Assignee: GLOBAL MEDISAFE HOLDINGS LIMITED, Newcastle NSW (AU)

(21) Appl. No.: 12/065,781

(22) PCT Filed: Mar. 20, 2006
(86) PCT No.: PCT/AU06/00372

§ 371 Date: Aug. 26, 2008

(51) Int. Cl. A61M 5/50; A61M 5/32

(52) U.S. Cl. 604/110; 604/196

(57) ABSTRACT

A single use safety retractable syringe (10) having a retractable needle (20) is disclosed. The syringe includes a hollow barrel (11), a plunger (13) which slideably fits in the barrel, and a piston (13) which fits on the forepart of the plunger. The syringe also includes an actuator mechanism (14), which is located in the forepart of the barrel. The actuator is able to be fitted through the open end of that forepart of the hollow barrel. The actuator mechanism has a stem (18) for supporting a needle hub (15). Means (24) are provided on the end of the plunger to engage the actuator and cause it to retract with the needle upon completion of the injection.

### TECHNICAL FIELD

This invention relates to a single use safety syringe. In particular it relates to a retractable needle for use in a single-use safety syringe, whereby the syringe user might be protected from injury by the needle after its use.

### BACKGROUND

The danger of injury and possible infection from the HIV or hepatitis B virus to medical practitioners using needles in the normal course of their business is well documented.

Further, persons who are in the habit of administering drugs to themselves run a severe risk of contacting either of the specified viruses, or indeed contacting other viruses if a needle, once used, is reused in an unsterilised form.

There have been many proposals aimed at reducing the number of so-called needle-stick injuries and various attempts have been made to provide a safe system for disposal of such needles once used, but such prior proposals have had deficiencies.

### OBJECT OF THE INVENTION

It is therefore an object of the present invention to provide a single use safety syringe which employs a retractable surgical needle and for permanently storing that surgical needle, once used, in a substantially safe manner, so as to reduce needle stick injuries as well as preventing re-usage of the syringe thereby preventing cross infections of diseases.

It is a particular object of the invention to ameliorate against some or all of the known disadvantages of prior art syringes and to provide a simple safety syringe having a retractable needle which renders the syringe unable to be re-used. At the very least, the invention provides an alternate means for accommodating the needle of a used syringe to protect against accidental injury arising from unwanted contact with the exposed needle once the syringe has been used.

### DISCLOSURE OF THE INVENTION

The invention in one broad form provides a single-use safety syringe having a retractable needle, the syringe including a hollow barrel, a plunger which slideably fits in the barrel, a piston which fits on the forepart of the plunger, and an actuator mechanism, which is located in the forepart of the barrel and which is able to be fitted therein through the open end of that forepart of the hollow barrel, the actuator mechanism having a stem for supporting a needle hub, and means located on the end of the plunger to engage the actuator and cause it to retract with the needle upon completion of the injection.

Where the actuator is located in the barrel, there is provided an inner flange on the barrel and a corresponding flange on the actuator mechanism, for fitment of the actuator from that open end of the barrel. The flanges are preferably shaped, for example having bevel surfaces on one side and right angled surfaces on the other, so that when the plunger is depressed (ie after the actuator has been thus fitted), the flange of the actuator mechanism locks against the corresponding inner flange of the barrel, for example by virtue of the opposing right angled surfaces coming into contact, so that in normal use the actuator will not be dislodged, Preferably a force of at least 98N may be endured. In this way, the actuator can not be dislodged from the barrel when the piston is depressed during an injection.

On the other hand the flanges need only withstand a lesser force than 98N in order to prevent the actuator from being withdrawn into the barrel when the needle of the syringe is inserted into the patient. Such force is preferably 49N. Indeed, in order that the actuator may be withdrawn into the barrel at the completion of the injection however, it is preferable that these opposing flange surfaces are beveled so that only a lesser force is needed, ie a force preferably between 49N and 98N.

At the point of contact between the actuator and the barrel there is preferably provided at least one “O” type piston seal, sufficient that there is no leakage between the actuator and the barrel during use.

It will be appreciated that the actuator will be hollow so that fluid contained in the barrel may pass through it and hence through the needle which it supports. The end of the stem of the actuator is preferably provided with at least one flexible finger member. The plunger is preferably provided with a solid stem having a shaped head so that when the injection is completed, the head of the plunger stem enters into the hollow stem of the actuator and passes through the end thereof and is thereby engaged with the actuator, the one or more fingers retaining the head of the plunger stem beyond the stem of the actuator. When the plunger is pulled back, exerting a force preferably between 49 and 98N pull force, the one or more fingers can not break off, and the actuator will be successfully pulled into the barrel.

In order to prevent premature engagement of the head of the plunger stem with the fingers of the actuator stem, a depth stop is preferably provided to prevent the plunger from thus engaging with the actuator before retraction of the needle is required. Preferably such depth stop is provided in the form of a pin located in a hole located transverse the shaft of the plunger and located thereon so as to prevent the plunger coming into contact with the actuator until the pin is depressed, thereby avoiding early unwarranted destruction of the syringe.

The present invention is therefore a single use safety syringe which includes the following components: a hollow barrel, one plunger which fits the barrel and one piston which fits the forepart of the plunger. There is an actuator which fits the forepart of the barrel. Preferably on the actuator there is located a standard 6:100 luer taper. This taper is designed to receive the hub of a needle utilising interference fit. Alternatively, in order to provide a more positive interconnection between the actuator and the needle hub, a screw thread including so-called luer-lock thread may be employed with advantage.

Between the barrel and the outer actuator surface, there is fitted at least one seal. At, the end of the barrel there is located at least one means for preventing complete depression of the plunger until the injection is complete and the syringe is to be rendered useless. At the forepart of the plunger there is located a pull back lock, preferably in the form of a head element for engagement with the forepart of the actuator's inner hole which is provided with flexible fingers, claws or the like, so that when the plunger is moved towards the actuator, the pull back hook in the forepart will enter into the actuator's engagement means easily. When the plunger is withdrawn, the actuator is pulled into the barrel, to realise the safe destruction thereof.

The safety syringe according to the preferred embodiments of the invention has the following advantages when compared with those of the existing market: firstly, it is provided with corresponding a flanges on the barrel and actuator to ensure that the actuator can not be dislodged and move back, an “O” type piston being provided between the actuator and the barrel, to make sure no liquid leaks out, and after injection, when the plunger is withdrawn the actuator can be pulled into the barrel easily, it can not be reused, achieving a truly safe syringe which is effectively destroyed; second, only need a minimal force to have the stem of the plunger enter into the flexible fingers o the stem of the actuator, thereby reducing the patient's pain which has been caused in injection in more recent times; third, the depth stop at the back-end of the plunger prevents the syringe being destroyed before it is used to give an injection through mis-operation causing premature destruction of the syringe.

Preferably the plunger is provided with a break point. Snapping off the plunger also renders the syringe both ineffective for subsequent use as well as providing additional safety in that there is no means by which the needle can be caused to re-emerge or extend from the syringe body.

Furthermore, it is preferable to provide means whereby the plunger can in fact be locked in position prior to being snapped off. This maybe achieved by utilising a sleeve or collar inserted in the end of the barrel, for example either by interference fit or by screwing it in place. A protrusion or lug may be provided on the plunger adjacent the break point and a passage for example like a thread may be provide in the sleeve or collar so that the plunger may be wound out through the collar or sleeve and locked by suitable means, eg flange or ratchet means so that when plunger is broken off at the break point there is no means by which it can be caused to re-enter the barrel as it is retained in the collar or sleeve.

### BRIEF DESCRIPTION OF THE DRAWINGS

The invention may be better understood from the following non-limiting description of preferred embodiments, in which:

FIG. 1 is an exploded view of a single-use safety syringe according to one embodiment of the invention;

FIG. 2 is a cross-sectional view of the assembled syringe of FIG. 1, showing the syringe prior to use with a cap covering and protecting the needle;

FIG. 3 is a cross-sectional view of the syringe of FIG. 2, showing the plunger drawn back, the barrel having been filled with injectable solution;

FIG. 4 is a cross-sectional view of the syringe of the previous figures, at the completion of injection, the plunger having engaged the actuator;

FIG. 5 is a cross-sectional view of the syringe of the previous figures, the plunger having been withdrawn so that the actuator and needle are safely withdrawn into the barrel of the syringe; and

FIG. 6 is a cross section of the syringe of the earlier figures, the plunger having been locked in the sleeve at the end of the barrel and broken off at the break point rendering the syringe safely destroyed.

### DETAILED DESCRIPTION OF THE DRAWINGS

Referring to FIG. I there is shown an exploded view of a syringe, generally referenced 10, according to a preferred embodiment of the invention.

The syringe includes a barrel 11, plunger 12, piston 13, actuator 14, needle hub 15 supporting a needle 20, “O” type piston 16, and depth stop 17. A cap 29 is provide to protect the needle prior to use.

An inner flange 21 is located in the foremost part of the barrel 11. A corresponding flange 22 is located on the actuator 14, for engagement of the actuator 14 when it is inserted into the barrel 11 from the open end of that foremost part of the barrel 11 The advantages of inserting the actuator 14 from that end of the barrel 11 lie in ease of assembly and less damage in situations where a needle hub 15 of a needle 20 is already fitted to the actuator 11; than would occur if the actuator were assembled by inserting it from the other (ie plunger) end of the syringe barrel 11.

Flexible spring fingers 23 are located on the end of the hollow stem 18 of the actuator 14. The solid stem 19 at the end of the plunger 13 is provided with a locking head 24. A hole 25 is located in the plunger 13 to accommodate the pin 7 which functions as a depth stop.

Break point 26 is provided on the plunger 13. The plunger also has flanges 28 which allow the plunger 13 to be twist locked in the twist lock sleeve 29 located in the end of the barrel 11.

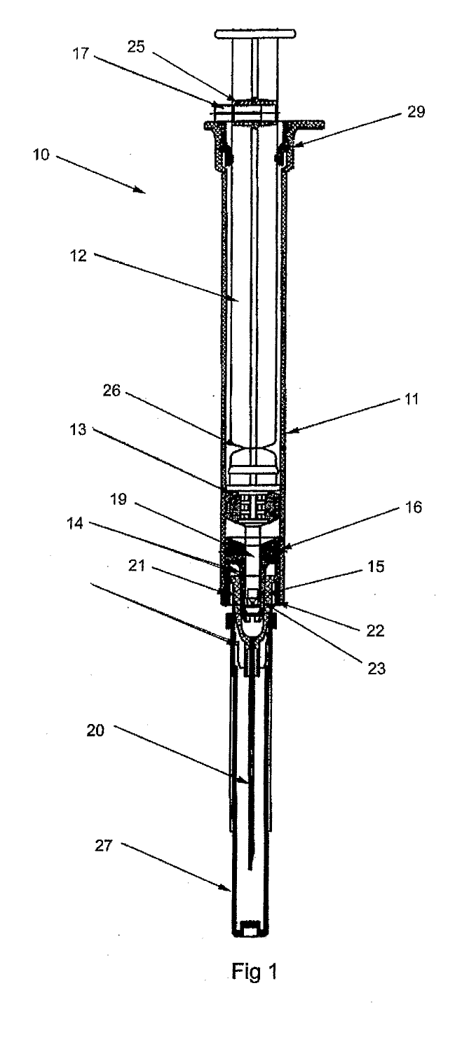

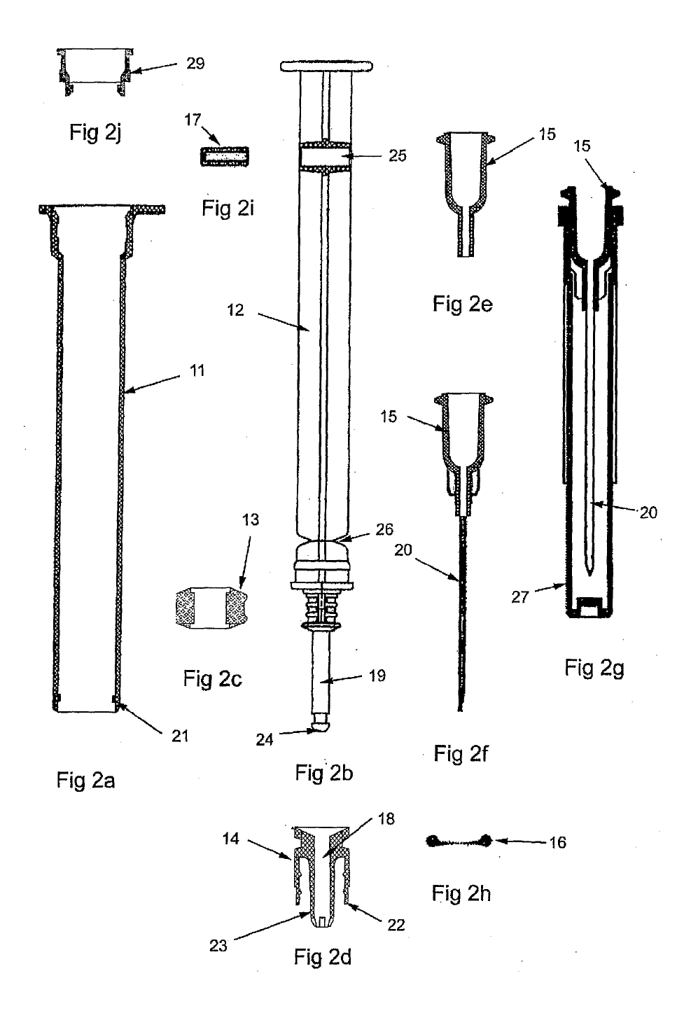

Referring generally to FIGS. 2 to 6, there is shown a syringe 10 which has the various parts illustrated as in FIG. 1, the same reference numerals being utilised throughout.

The safety syringe 10 according to the invention possesses the following characters: At the junction between the actuator 14 and barrel 11 there is located a sealing member 16 in the form of an “O” ring or the like. There is also located corresponding flanges 22, 21 on the actuator 14 and on the inner surface of the barrel, 11 respectively. At each edge of the actuator flange 22 there is formed a bevel and a right angle respectively, the bevel and right angle correspond with a bevel and a right angle located on the flange 21 inside the barrel 11. In essence the orientation of the respective shapes of the two flanges 21, 22 means that in use, normal pressure as exerted during an injection will not cause the actuator to be dislodged outwardly or indeed inwardly. Indeed the use of the depth stop 17, prior to its being pressed in will ensure that this can't happen accidentally either. However, the use of the locking head 24 which engages the fingers 23 is in fact sufficient to easily draw the actuator 14 back, once the locking head 24 of the plunger 13 has been allowed to engage the actuator 14 by pressing in stop 17.

On the plunger 13 there is provided a break point 26, so that the plunger 13 can be broken off easily along the break point 26. At the back-end of plunger 13 there is located a depth stop 17 which can be pushed in, but which contacts the end of the barrel 11, until such time as it is pushed in so as to prevent the plunger 13 from being fully depressed and hence engaging with the actuator 14.

In FIG. 2, the needle is protected-by a cap 27 prior to use. The plunger 13 is prevented from engaging with actuator 14 by virtue of depth stop 17 which is in the out position.

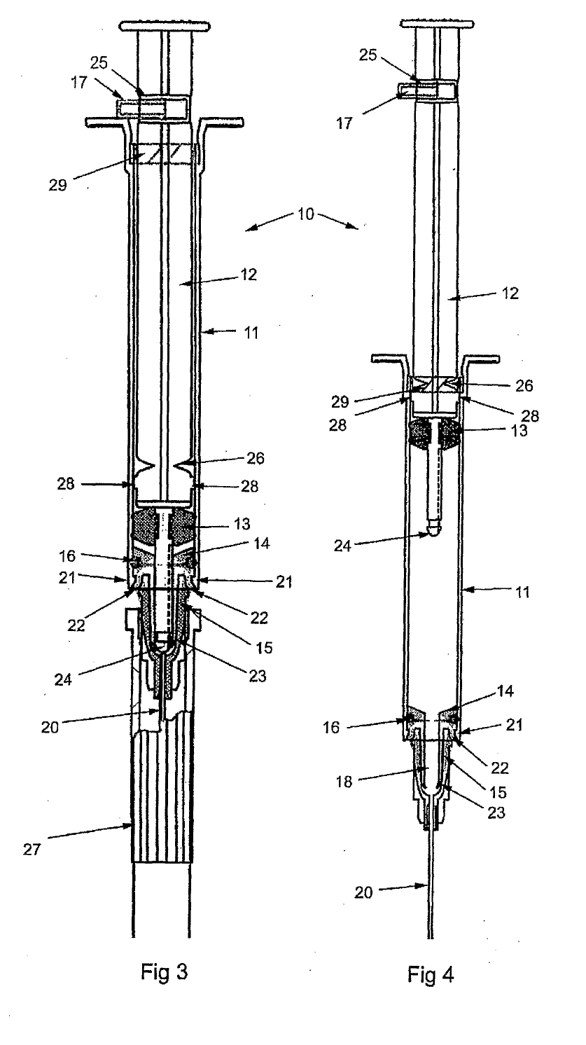

In use the cap 27 is removed to reveal the needle 20 and the needle 20 is inserted into the fluid to be used for the injection (such as in a vial—not shown). The plunger 13 is then withdrawn as in FIG. 3 to take fluid into the barrel 11.

The injection is then performed after the depth stop 17 has been pushed in so that the plunger 13 now engages with the actuator 14 as in FIG. 4.

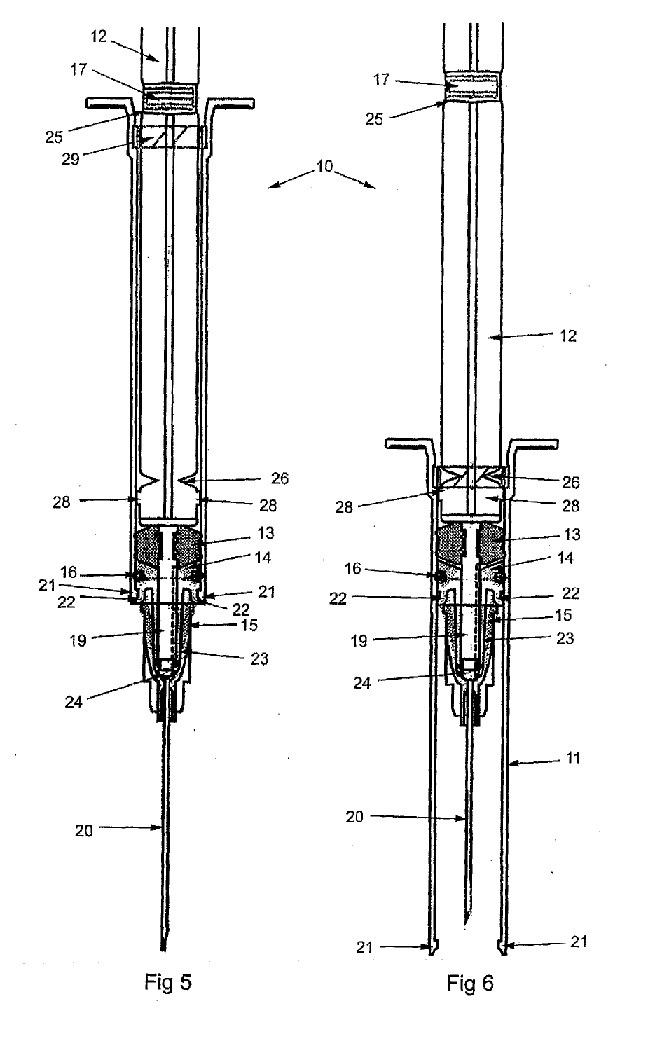

At the end of the injection, the locking head 24 will have entered the hollow stem 18 of the actuator 14 and beyond into the flexible fingers 23 so that it is retained therein. Upon withdrawing the plunger 13 once more, the actuator 14 and needle hub/needle assembly 15,20 are caused to be withdrawn into the barrel 11 of the syringe 10 as shown in FIG. 5.

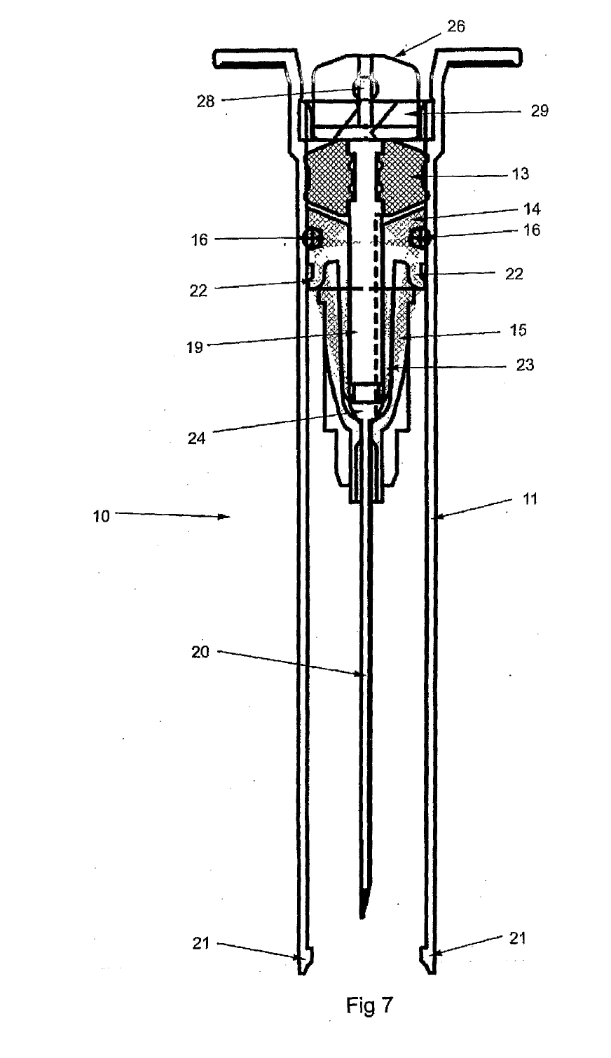

As shown in FIG. 6, the flanges 28 located on the plunger 13 are twisted through the twist lock sleeve 29 fitted in the end of the barrel. Breaking off the plunger 13 at the break point 26 renders the syringe 10 totally safe and useless.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. A single use safety retractable syringe having a retractable needle, the syringe including a hollow barrel, a plunger which slideably fits in the barrel, a piston which fits on the forepart of the plunger, and an actuator mechanism, which is located in the forepart of the barrel and which is able to be fitted therein through the open end of that forepart of the hollow barrel, the actuator mechanism having a stem for supporting a needle hub, and means located on the end of the plunger to engage the actuator and cause it to retract with the needle upon completion of the injection, wherein the stem of the actuator is hollow and provided with at least on flexible finger member and wherein in order to prevent premature engagement of the head of the plunger stem with the fingers of the actuator stem, a depth stop is provided to prevent the plunger from thus engaging with the actuator before retraction of the needle is required, the depth stop being provided in the form of a pin located in a hole located transverse the shaft of the plunger and located thereon so as to prevent the plunger coming into contact with the actuator until the pin is depressed, thereby avoiding early unwarranted destruction of the syringe.

2. A single use safety retractable syringe according to claim 1, wherein at the point where the actuator is located in the barrel, there is provided an inner flange on the barrel and a corresponding flange on the actuator mechanism.

3. A single use safety retractable syringe according to claim 2, wherein the respective flanges are shaped, having bevel surfaces on one side and right angled surfaces on the other, so that when the plunger is depressed, the flange of the actuator mechanism locks against the corresponding inner flange of the barrel, by virtue of the opposing right angled surfaces coming into contact, so that in normal use the actuator will not be dislodged.

4. A single use safety retractable syringe according to claim 3, wherein a force of at least 98N may be endured, so that the actuator can not be dislodged from the barrel when the piston is depressed during an injection under normal use conditions.

5. A single use safety retractable syringe according to claim 3, wherein the flanges need only withstand a lesser force than 98N in order to prevent the actuator from being withdrawn into the barrel when the needle of the syringe is inserted into the patient.

6. A single use safety retractable syringe according to claim 5, wherein the lesser force is preferably 49N.

7. A single use safety retractable syringe according to claim 5 wherein the opposing flange surfaces are beveled so that only a lesser force is needed, ie a force preferably between 49N and 98N.

8. A single use safety retractable syringe according to claim 1 wherein at the point of contact between the actuator and the barrel there is provided at least one “O” type piston, sufficient that there is no leakage between the actuator and the barrel during use.

9. A single use safety retractable syringe according to claim 1 wherein the plunger is provided with a solid stem having a shaped head so that when the injection is completed, the head of the plunger stem enters into the hollow stem of the actuator and passes through the end thereof and is thereby engaged with the actuator, the one or more fingers retaining the head of the plunger stem beyond the stem of the actuator.

10. A single use safety retractable syringe according to claim 9, wherein when the plunger is pulled back, exerting a force between 49 and 98N pull force, the one or more fingers can not break off, and the actuator will be successfully pulled into the barrel.

11. A single use safety retractable syringe according to claim 1 wherein on the actuator there is located a standard 6:100 luer taper to receive the hub of a needle utilising interference fit.

12. A single use safety retractable syringe according to claim 1, wherein in order to provide a more positive interconnection between the actuator and the needle hub, a screw thread including a so-called luer-lock thread is employed.

13. A single use safety retractable syringe according to claim 1 wherein the plunger is provided with a break point, so that the plunger is snapped off at completion of the injection rendering the syringe both ineffective for subsequent use as well as providing additional safety in that there is no means by which the needle can be caused to re-emerge or extend from the syringe body.

14. A single use safety retractable syringe according to claim 13 wherein means are provided whereby the plunger can be locked in position prior to being snapped off.

15. A single use safety retractable syringe according to claim 14 wherein a sleeve or collar is inserted in the end of the barrel by interference fit or by screwing it in place and one or more protrusions or lugs are provided on the plunger adjacent the break point and a passage like a thread is provided in the sleeve or collar so that the plunger may be twisted or wound out through the collar or sleeve and locked by suitable means, including flange or ratchet means so that when plunger is broken off at the break point there is no means by which it can be caused to enter the barrel again as it is retained in the collar or sleeve.

16. (canceled)
